(19) United States
(12) Patent Application Publication
(10) Pub. No.: US 2010/0094129 A1
(43) Pub. Date: Apr. 15, 2010

(54) ESOPHAGIAL VISUALIZATION DEVICE

(76) Inventors: Frank Marchilinski, Bala Cynwyd, PA (US); Sen Ji, La Canada, CA (US)

Correspondence Address: Pearl Cohen Zedek Latzer, LLP, 1500 Broadway, 12th Floor, New York, NY, 10036, US

(21) Appl. No.: 12/520,029

(22) PCT Filed: Dec. 18, 2007
(86) PCT No.: PCT/US07/25791

§ 371 Date: Dec. 3, 2009

Related U.S. Application Data
(60) Provisional application No. 60/875,797, filed on Dec. 20, 2006.

(51) Int. Cl. A61B 6/00; A61B 18/18

(52) U.S. Cl. 600/431; 606/41

(57) ABSTRACT

This invention relates to a device for continuously visualizing the esophagus during atrial finrillation ablation procedure to reduce the risk of developing atrio-esophagial fistula as the result of atrial fibrillation ablation.

### FIELD OF INVENTION

This invention is directed to devices and methods for providing continuous visualization contrast during a medical procedure. Specifically, the invention is directed to a device for continuously visualizing the esophagus during atrial finrillation ablation procedure to reduce the risk of developing atrio-esophagial fistula as the result of atrial fibrillation ablation.

### BACKGROUND OF THE INVENTION

Atrial fibrillation (a-fib) is the most commonly encountered arrhythmia in daily clinical practice. Data suggest pulmonary veins are involved in the initiation and maintenance of atrial fibrillation. Isolation of the veins using radiofrequency energy is becoming the most effective means in curing a-fib However, one of the potential complications during a-fib ablation is the formation of an often fatal atrio-esophageal fistula. The occurrence of this complication is due to several reasons. One such reason is the proximity of the esophagus to the left atrium. Two structures are separated by a layer of tissue of several millimeters. Another is that the esophagus is a structure constantly moving due to peristalsis. Therefore, a device which can be used to continuously visualize the esophagus in real time through out the procedure would help avoid energy delivery in regions of the left atrium directly opposite to the esophagus and hence the fatal complication.

### SUMMARY OF THE INVENTION

In one embodiment, the invention provides a device for providing a visualizing contrast during a medical procedure comprising: a proximal injection port; a radio-opaque distal flexible container; and a tube disposed therebetween.

In another embodiment, provided herein is a method of providing a continuous visualizing contrast for the esophagus of a subject during a medical procedure, comprising the steps of: nasopharingially intubating the subject with a device comprising a proximal injection port, a distal flexible container, and a tube disposed therebetween, whereby said flexible container and said tube are comprised of a radio-opaque material; positioning the distal end of the flexible container behind the heart of the subject; and inflating the flexible container, whereby inflating the flexible container results in the filling of the esophagus, thereby providing visualization contrast.

In one embodiment, provided herein is a method of reducing risk of developing atrio-esophageal fistula in a subject, resulting from an atrial fibrillation ablation procedure, comprising the step of continuously visualizing the esophagus during the atrial fibrillation ablation.

In another embodiment, provided herein is a system for providing continuous visualization contrast in a subject during imaging of a medical procedure comprising: an imaging means; a device providing continuous contrast for the imaging means, said device comprising: a proximal injection port; a radio-opaque distal flexible container; and a tube disposed therebetween.

### BRIEF DESCRIPTION OF THE DRAWINGS

FIG. 1 shows the injection port (1) at the proximal end of the injection tube (2), leading to the visualization pouch (3). An insertion stylet (4) is used to insert the visualization pouch through the nostrils of the patient to the esophagus, wherein a medium is inserted through the injection port (1), inflating the visualization tube (3) thereby filling the subjects esophagus with a radio-opaque background for continuous visualization of the esophagus during a-fib ablation.

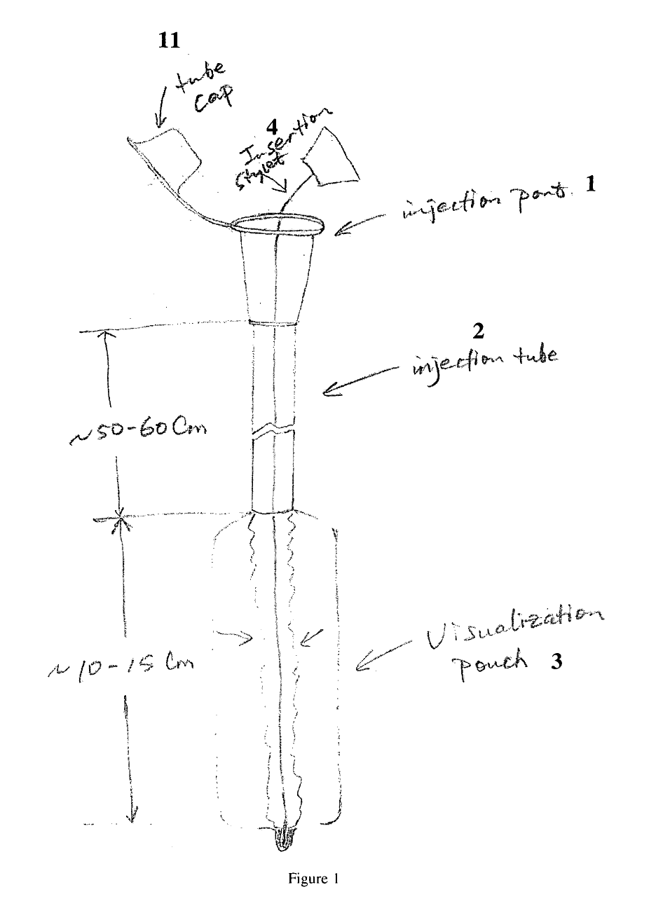

FIG. 2 shows the prototype of the device made after modification and improvement of original design shown in FIG. 1, comprising two ports for injection (1) and suction (6), a double-lumened tube for injection and suction, (21) a visualization pouch for holding and circulating contrast fluid (3) and a thermo-filament for temperature-monitoring (7).

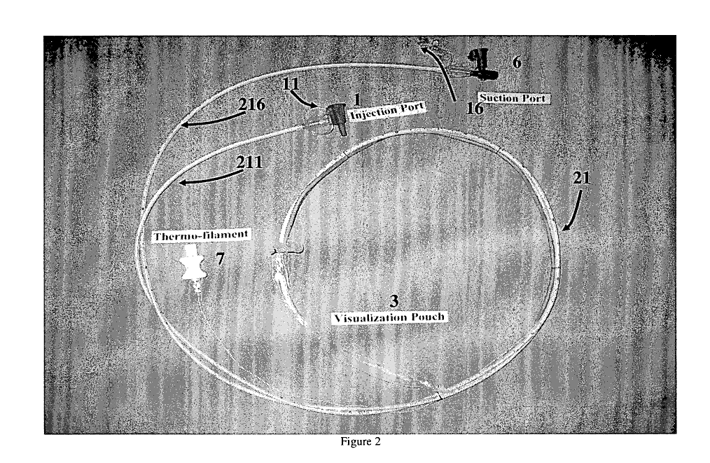

### DETAILED DESCRIPTION OF THE INVENTION

This invention relates in one embodiment to devices and methods for providing continuous visualization contrast during a medical procedure. Specifically, the invention is directed to a device for continuously visualizing the esophagus during atrial finrillation ablation procedure to reduce the risk of developing atrio-esophagial fistula as the result of atrial fibrillation ablation.

In one embodiment, an esophageal visualization pouch is provided, which allows the visualization of the esophagus without the risk of causing aspiration of oral contrast during medical procedures such as atrial fibrillation ablation.

In another embodiment, an esophageal cooling pouch with temperature-monitoring filament is provided, which allows the control and monitor of the temperature in the esophagus to avoid the risk of collateral thermo-damage of the esophagus during a-fib ablation.

In one embodiment, catheter ablation around the pulmonary vein region of the posterior left atrium is used to cure atrial fibrillation. With the posterior wall of the left atrium in close proximity to the esophagus, in certain embodiments, radiofrequency ablation procedures substantially elevate the temperature within the esophageal lumen causing thermal damage. In other embodiments, the risk of developing Atrial—esophageal fistulas from this thermal injury and subsequent necrosis is reduced using the devices and systems described herein.

According to this aspect of the invention and in one embodiment, provided herein is a device for providing a visualizing contrast during a medical procedure comprising: a proximal injection port (1); a radio-opaque distal flexible container (3); and a tube disposed therebetween (2).

In one embodiment, the ability of a chemical species to absorb X-rays is related directly to its atomic number and is approximated by the relationship

m=kl³Z⁴ (EQN. 1)

Where −m is the absorption coefficient;
−l is the wavelength of the incident X-ray:
−Z is the atomic number of the absorbing species: and
−k is the proportionality constant.

Iodine and bromine atoms, because of their high mass, scatter X-rays and impart radio-opaqueness. In one embodiment, the materials used to construct the distal flexible container, are material with high absorption coefficient. In another embodiment, the radio-opaque materials involve compounding materials like barium sulfate (i.e., Ba SO.sub.4) into the objects; or plating/ion sputtering silver or gold onto the device and systems described herein. In another embodiment, the devices and systems described herein, are filled with barium or bismuth radio-opaque compounds. In another embodiment, radio-opaque paints and inks with barium sulfate or silver powders are physically trapped in the devices and systems described herein. A person skilled in the art would recognize that the invention described herein, encompasses any suitable radio-opaque material capable of either being molded into the devices and systems described herein, or incorporated into the devices and systems described herein.

In another embodiment, the term “atrial fibrillation” (a-fib) refers to a rapid, irregular heart rhythm caused by abnormal electrical signals from the upper chambers of the heart (atrium). A-fib may increase the heart rate in one embodiment, to and in excess of between about 100 to 175 beats per minute. As a result and in another embodiment, the atria quiver rather than contract normally, resulting in blood pooling in the atria, the formation of blood clots and strokes.

In one embodiment, the devices and systems provided herein comprise two ports for injection (1) and suction (6), a double-lumened tube for injection and suction, (21) a visualization pouch (3) and a thermo-filament for temperature-monitoring (7). In another embodiment, the two ports (1, 6) are made to accommodate the tip of a regular syringe or to connect to an IV or peristaltic pump, one port for injection (1) and the other port for suction (6). In one embodiment, the injection and suction ports (1, 6 respectively), will have different colors, blue for injection and white for suction. The double-lumen tube (21), is made in another embodiment from medical grade polyurethane with centimeter marks, and in another embodiment, contain an injection lumen (211) and a suction lumen (216). The injection lumen (211) will reach the distal end of the visualization pouch (3), but the suction lumen (216), ends at the proximal end of the visualization pouch (3), to ensure adequate circulation. In one embodiment, the visualization pouch (3), is thin and highly flexible to accommodate the shape of the esophagus, while in another embodiment being highly resistant to pressure. In one embodiment, the visualization pouch is made of Latex coated with a radio-opaque dye. The thermal filament (7) is embedded in one embodiment, in the middle of the double-lumen tube (21) and extended to the surface of the middle part of the visualization pouch facing the left atrium (3). Using the devices described herein, the temperature of the esophagus can be measured continuously and adjusted by changing the temperature of the fluid circulating between the injection port (1) and suction port port and through the visualization pouch (2).

In one embodiment, the injection port (1) provided in the devices and systems and used in the methods provided herein, further comprise a cap (11). In another embodiment, both the injection and suction ports (1 and 6 respectively), further comprise a cap (11 and 16 respectively). In one embodiment, the injection tube (2), or the double lumen injection tube (21) described hereinabove have graduated indicia thereon. The graduated indicia is adapted in one embodiment to measure volume (e.g. μl, ml), or distance (in, cm, mm). In one embodiment, the graduated indicia is marked on the injection tube (2), or the double lumen injection tube (21), with a radio-opaque dye.

In one embodiment, the device used in the systems and methods described herein further comprises a soft stylet (4) inserted to the end of the injection tube (1) to facilitate the insertion process. The visualization pouch (3) is deflated and packed around the stylet (4) and the injection tube (1). In another embodiment, insertion of the device into the esophagus is done in the same manner as conventional nasogastric feeding tube. In one embodiment, the subject is in a sitting position. The tube is inserted into one of the nostrils, is directed posteriorly using the guiding stylet (4), and then advanced into nasopharynx, allowing the tip to seek its own passage while the subject is swallowing sips of water. Once into the esophagus, the tube (2) is positioned under a fluoroscope guidance, with the tip at the level of the diaphragm and the visualization pouch (3) behind the heart. Once the pouch (3) is positioned properly, it is inflated with normal saline containing oral radio-opaque contrast dye, to fill the esophagus. The tube (2) is then capped (11) if visualization is desirable but not temperature control. In one embodiment, the normal saline containing oral contrast is circulated continuously to obtain a desired temperature while maintaining continuous visualization of the esophagus during the medical procedure. The pouch (3) is left in one embodiment, in the esophagus through the procedure and the position of the esophagus is then visualized constantly. At the end of the procedure (a-fib ablation) and in another embodiment, normal saline mixed with oral contrast will be withdrawn from the pouch, causing decompression of the pouch (3). The tube will then be pulled out. In one embodiment, the devices and systems described hereinabove, are used in the methods described herein.

In one embodiment, the devices and systems described herein, which are used in the methods described herein, further comprise a soft stylet or a stiffening wire as a guide member. In one embodiment, the devices and systems described herein further comprise stiffening stylets or a wire guide that are placed into the lumens of the devices described herein. In certain embodiments. the guide wires impart column strength and stiffness to the devices described herein to enable its transesophagial advancement to its final location.

In one embodiment, the devices used in the systems and methods described herein have flexibility to navigate the twists and turns of the access pathway, as well as sufficient column strength in the proximal segment thereof to be pushed through the access pathway alone or over a guide wire or through a lumen, such as the esophagus in one embodiment and the capability of orienting the distal visualization pouch (3) and in another embodiment, the thermocouple sensor (7) of the distal segment in alignment with the heart at the accessed site so that an anatomical structure such as the esophagus in one embodiment, can be continuously visualized. In one embodiment, the elongated device body also resists kinking and is capable of being advanced through access pathways that twist and turn, sometimes abruptly at acute angles, such as in another embodiment, the nostril. In another embodiment, a lubricant is used on the surface of the said device for its passage through the naso-pharyngial pathway and the esophagus.

In one embodiment, the stylet or guide wire is removable. The removable stylet can be a steerable stylet of the types described in commonly assigned U.S. Pat. Nos. 5,873,842 and 6,146,338, for example, all which are incorporated herein by reference in their entirety.

The stylet comprises in one embodiment, a length of stainless steel wire extending from a proximal stylet manipulating means, such as a loop or a nob, operably coupled to the proximal end of a stainless steel guide wire in another embodiment, then to the stylet guide wire distal end attached to the distal end of the visualization pouch (3). In one embodiment, the stylet wire has a diameter of between about 0.022 inches (0.56 mm) to about 0.08 inches (2.0 mm) In one embodiment, the stylet or guiding wire is manually shaped to impart a curve in its distal segment and inserted through the injection lumen (211) to the visualization container (3). In one embodiment, the flexible container (3), is packed around the stylet or stiffening wire guide member.

In one embodiment, the radio-opaque material of the devices used in the systems described herein, is a bio-compatible polymer. The term “bio-compatible”, refers in one embodiment to a material that does not invoke a significant inflammatory or toxic response. In one embodiment, the bio-compatible polymer is polyurethane, latex, hydrophillic silicone, Santoprene, C-flex, Kraton, neoprene or other similarly bio compatible synthetic polymers.

In one embodiment, the devices described hereinabove, are used in the methods described herein. According to this aspect of the invention and in one embodiment, provided herein is a a method of providing a continuous visualizing contrast for the esophagus of a subject during a medical procedure, comprising the steps of: nasopharingially intubating the subject with a device comprising a proximal injection port (1), a distal flexible container (3), and a tube disposed therebetween (2), whereby said flexible pouch (3) and said tube (2) are comprised of a radio-opaque material; positioning the distal end of the flexible pouch (3) behind the heart of the subject; and inflating the flexible container, whereby inflating the flexible container results in the filling of the esophagus, thereby providing visualization contrast. In one embodiment the flexible container is inflated using a solution containing a contrast agent, such as amidotrizoic acid in certain embodiments.

In another embodiment, described herein is a method of providing a continuous visualizing contrast for, and controlling and monitoring the temperature of the esophagus of a subject during a medical procedure, comprising the steps of: nasopharingially intubating the subject with a device comprising a proximal injection and suction port, a distal flexible container, a double-lumen tube disposed therebetween, and a thermofilament attached, whereby said flexible container and said tube are comprised of a radio-opaque or non-radio-opaque material; positioning the distal end of the flexible container behind the heart of the subject; and inflating the flexible container, whereby inflating the flexible container results in the filling of the esophagus, thereby providing visualization contrast; circulating contrast material pre-cooled to certain temperature through the injection port, distal flexible container and suction port, whereby this process results in control of the temperature of the esophagus, thereby preventing thermo-damage to the esophagus; and monitoring the temperature of the esophagus, thereby providing information to adjust the temperature of the circulating contrast.

The esophagus begins as an extension of the pharynx in the back of the oral cavity. It then courses down the neck behind the trachea and the left atrium through the thoracic cavity, and penetrates the diaphragm to connect with the stomach in the abdominal cavity. It consists of four layers, including adventitia, muscularis, submucosa and mucosa. It is a flat tubular structure when not delivering food boluses. It undergoes peristalsis during the passage of food. As mentioned above, the esophagus is separated from the left atrium by a thin layer of tissue of several millimeters and its position may change due to peristalsis. In one embodiment, the methods described herein are used to provide contrast in the esophagus, for a medical procedure requiring the visualization of the esophagus relative to the heart, throughout the medical procedure in a way that is insensitive to peristalsis.

In one embodiment, the medical procedure requiring the continued visualization of the esophagus using the methods described herein, is aterial fibrillation ablation. Atrio-esophageal fistula is an often fatal complication resulting in one embodiment from intraoperative radiofrequency ablation of a-fib, with an incidence reported to be as high as 1%, or in another embodiment from percutaneous radiofrequency catheter ablation in the left atrium. The esophagus lies against the groove formed by the left- and right-sided pulmonary veins (PVs), and the posterior left atrium (LA) is targeted in certain embodiments during elimination of non-PV arrhythmogenic foci, extraostial PV isolation, PV antrum disconnection, circumferential PV ablation, or wide-area circumferential ablation. In one embodiment, continuous esophagial visualization using the methods and systems described herein, is effective in reducing atrioesophageal fistulas resulting from these procedures.

In one embodiment, the step of positioning the visualization or cooling pouch behind the subject's heart, is done under fluoroscope guidance. In one embodiment, a fluoroscope is employed to provide visual confirmation that the stylet or guide wire, or thermosensor is positioned appropriately within the visualization container, during or after the positioning of the devices described herein, that are used in the methods described herein, within the esophagus of the subject.

In one embodiment, the methods of providing continuous visualization of the esophagus as described hereinabove, are used to reduce the risk of developing aterio-esophageal fistula. According to this aspect of the invention and in another embodiment, provided herein is a method of reducing risk of developing atrio-esophageal fistula in a subject, resulting from an atrial fibrillation ablation procedure, comprising the step of continuously visualizing the esophagus during the atrial fibrillation ablation.

In one embodiment, the step of continuously visualizing, cooling, temperature monitoring or their combination of the esophagus in the methods described herein, is achieved by providing a continuous visualizing contrast for the esophagus during the aterial fibrillation ablation.

In one embodiment, the devices and methods described hereinabove are used in are performed by the systems described herein. According to this aspect of the invention and in one embodiment, provided herein is a system for providing continuous visualization contrast, cooling, temperature monitoring or their combination in a subject during imaging of a medical procedure comprising: an imaging means; a device providing continuous contrast for the imaging means, said device comprising: a proximal injection port; a radio-opaque distal flexible container; and a tube disposed therebetween, wherein continuous visualization is obtained by nasopharingially intubating the subject with a device comprising a proximal injection port, a distal flexible container, and a tube disposed therebetween, whereby said flexible container and said tube are comprised of a radio-opaque material; positioning the distal end of the flexible container behind the heart of the subject; and inflating the flexible container, whereby inflating the flexible container results in the filling of the esophagus, thereby providing visualization contrast.

In one embodiment, the device used in the methods and systems described herein, comprises a tube that is a double-lumen tube with the injection lumen reaching the distal end of the container and further comprising a suction lumen reaching only the proximal end of the container such that the methods of visualizing, cooling, temperature monitoring or their combination as described herein, further comprise the steps of circulating contrast-fluid in the container; and monitoring temperature in the esophagus, whereby circulating contrast fluid results in control of the temperature of the esophagus wall.

The devices and methods described hereinabove are used and are performed by the systems described herein. According to this aspect of the invention and in one embodiment, provided herein is a system for providing continuous visualization contrast, colling and temperature-monitoring in a subject during imaging of a medical procedure comprising: an imaging means; a device providing continuous contrast for the imaging means, said device comprising: a proximal injection and suction port; a radio-opaque distal flexible container; a tube disposed therebetween and thermo-sensor, wherein continuous visualization is obtained by nasopharingially intubating the subject with a device comprising a proximal injection and suction port, a distal flexible container, a tube disposed therebetween; and a temperature sensor, whereby said flexible container and said tube are comprised of a radio-opaque material; positioning the distal end of the flexible container behind the heart of the subject; and inflating the flexible container, whereby inflating the flexible container results in the filling of the esophagus, thereby providing visualization contrast; circulating pre-cooled contrast fluid in the flexible container results cooling of the esophagus, thereby preventing the collateral thermal damage from a-fib ablation; and recording real temperature results in monitoring the temperature of the esophagus.

In another embodiment, described herein is a system of providing continuous temperature-control of the esophagus in a subject during a medical procedure comprising: a cooling means, a device providing continuous circulation of pre-cooled contrast fluid for control of the temperature of the esophagus, said device comprising: a proximal injection and suction port; a radio-opaque distal flexible container; a double lumen tube disposed therebetween; and thermo-filament attached.

In another embodiment, described herein is a system for providing continuous temperature-monitoring of the esophagus in a subject during a medical procedure comprising: a temperature monitoring means, a device providing continuous monitoring of the temperature in the esophagus, said device comprising: a proximal injection and suction port; a radio-opaque distal flexible container; a double lumen tube disposed therebetween; and thermo-filament attached.

In one embodiment the imaging means is fluoroscopy, Transesophageal echocardiography, magnetic resonance imaging, computer aided tomography, or intracardiac echocardiography (ICE) and the like.

The term “about” as used herein means in quantitative terms plus or minus 5%, or in another embodiment plus or minus 10%, or in another embodiment plus or minus 15%, or in another embodiment plus or minus 20%.

The term “subject” refers in one embodiment to a mammal including a human in need of therapy for, or susceptible to, a condition or its sequelae. The subject may include dogs, cats, pigs, cows, sheep, goats, horses, rats, and mice and humans. The term “subject” does not exclude an individual that is normal in all respects.

Having described preferred embodiments of the invention with reference to the accompanying drawings, it is to be understood that the invention is not limited to the precise embodiments, and that various changes and modifications may be effected therein by those skilled in the art without departing from the scope or spirit of the invention as defined in the appended claims.

1. A device for providing a visualizing contrast during a medical procedure comprising:
a. a proximal injection port;
b. a radio-opaque distal flexible container; and
c. a tube disposed therebetween.

2. The device of claim 1, wherein the injection port further comprises a capping means.

3. The device of claim 1, wherein the injection port is adapted to accommodate a syringe tip.

4. The device of claim 1, wherein the injection tube has graduated indicia thereon.

5. The device of claim 4, wherein the graduated indicia is radio-opaque.

6. The device of claim 1, wherein the flexible container is a balloon.

7. The device of claim 1, further comprising a soft stylet or a stiffening wire guide member.

8. The device of claim 7, wherein the flexible container is packed around the stylet or stiffening wire guide member.

9. The device of claim 1, wherein the medical procedure is atrial fibrillation ablation.

10. The device of claim 1, wherein the radio-opaque material is a bio-compatible polymer.

11. The device of claim 10, wherein the bio-compatible material is hydrophillic silicone, Santoprene, polyurethane, C-flex, Kraton, latex, or neoprene.

12. The device of claim 1, wherein the tube is comprised of a radio-opaque material.

13. The device of claim 1, wherein the tube is a double-lumen tube with the injection lumen reaching the distal end of the container and further comprising a suction lumen reaching only the proximal end of the container.

14. The device of claim 13, further comprising a thermo-sensor embeded in the wall of the double-lumen tube and the pouch.

15. The device of claim 13, wherein the suction lumen has a suction port at its proximal end.

16. The device of claim 15, wherein the suction port is adapted to accommodate a syringe tip.

17. A method of providing a continuous visualizing contrast for the esophagus of a subject during a medical procedure, comprising the steps of:
a. nasopharyngeally intubating the subject with a device comprising a proximal injection port, a distal flexible container, and a tube disposed therebetween, whereby said flexible container and said tube are comprised of a radio-opaque material;
b. positioning the distal end of the flexible container behind the heart of the subject; and
c. inflating the flexible container, whereby inflating the flexible container results in the filling of the esophagus, thereby providing visualization contrast.

18-21. (canceled)

22. The method of claim 17, whereby the medical procedure is atrial fibrillation ablation.

23-37. (canceled)

38. A method of reducing risk of developing atrio-esophageal fistula in a subject, resulting from an atrial fibrillation ablation procedure, comprising the step of continuously visualizing the esophagus during the atrial fibrillation ablation.

39-40. (canceled)

41. A system for providing continuous visualization contrast in a subject during imaging of a medical procedure comprising:
a. an imaging means;
b. a device providing continuous contrast for the imaging means, said device comprising:
i. a proximal injection port;
ii. a radio-opaque distal flexible container; and
iii. a tube disposed therebetween.

42-54. (canceled)
